(19) United States
(12) Patent Application Publication
(10) Pub. No.: US 2026/0224453 A1
(43) Pub. Date: Aug. 6, 2026

(54) REDUCTION OF DISCOLORATION IN TEETH TREATED WITH ANTIMICROBIAL AGENTS

(71) Applicant: Regents of the University of Minnesota, Minneapolis, MN (US)

(72) Inventors: Samuel Brian Lossef, Washington, DC (US); Hooi Pin Chew, Bloomington, MN (US)

(21) Appl. No.: 19/531,104

(22) Filed: Feb. 5, 2026

Related U.S. Application Data
(60) Provisional application No. 63/754,368, filed on Feb. 5, 2025.

(51) Int. Cl. A61K 8/21; A61K 8/23; A61Q 11/00

(52) U.S. Cl. CPC A61K 8/21; A61K 8/23; A61Q 11/00; A61K 2800/81; A61K 2800/92

(57) ABSTRACT

A method for removing dental discoloration includes applying a laser treatment to a tooth, such as at a particular wavelength or within a particular range of wavelengths. The tooth includes one or more discolored silver compounds generated from a silver-containing therapeutic agent treatment, such as silver diamine fluoride treatment. Application of the laser treatment selectively breaks the chemical bonds of the one or more discolored silver compounds.

### CROSS-REFERENCE TO RELATED APPLICATIONS

This application claims the benefit of U.S. Provisional Patent Application Serial No. 63/754,368, filed February 5, 2025, and entitled “REDUCTION OF DISCOLORATION IN TEETH TREATED WITH ANTIMICROBIAL AGENTS,” which is incorporated by reference herein in its entirety.

### TECHNICAL FIELD

The present disclosure relates to devices and methods for reducing discoloration following treatment with an antimicrobial agent, such as silver diamine fluoride treatment.

### BACKGROUND

Silver diamine fluoride (SDF) is a silver-containing topical re-mineralizing agent that is effective at arresting dental caries. SDF includes a combination of silver ions that have anti-microbial properties, amine groups that neutralize acid, and fluoride ions that promote remineralization.

### SUMMARY

This disclosure describes devices and methods for reducing discoloration in teeth treated with SDF. A surface portion of a tooth treated with a silver diamine fluoride (SDF) treatment includes silver compounds, such as silver oxide or silver sulfide, that are generated from a reaction between silver ions and oxygen- and sulfur-containing compounds. To reduce the discoloration caused by these silver compounds, a laser treatment is applied to the tooth to selectively break the chemical bonds of the silver compounds. For example, laser energy applied to the tooth may have a particular wavelength or range of wavelengths that is selectively absorbed by the silver compounds and converts the silver compounds to more neutrally-colored elements or compounds, such as elemental silver and sulfur dioxide. The laser energy may be maintained at a fluence and pulse frequency that maintains a temperature of the tooth below thresholds that may damage the tooth. Following laser treatment, the tooth may be optionally coated with an additional coating, such as a glass ionomer cement (GIC) coating, to additionally mask residual discoloration of the tooth. By reducing discoloration caused by silver compounds, SDF treatment may be more aesthetically viable for a greater number of patients, and particularly patients who may not otherwise consider SDF treatment.

In one example, a method for reducing or removing dental discoloration includes applying a laser treatment to a tooth. The tooth includes discolored silver compounds generated from a silver-containing therapeutic agent treatment, such as silver diamine fluoride treatment. Application of the laser treatment selectively breaks the chemical bonds of the silver compounds.

In another example, a system for reducing or removing dental discoloration includes a laser treatment system configured to apply a laser treatment to a tooth. The tooth includes discolored silver compounds generated from a silver-containing therapeutic agent treatment, such as silver diamine fluoride treatment. Application of the laser treatment selectively breaks the chemical bonds of the silver compounds.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a flowchart illustrating an example method for removing dental discoloration.

FIG. 2A is a front view diagram illustrating a tooth prior to treatment with silver diamine fluoride.

FIG. 2B is a front view diagram illustrating a tooth after treatment with silver diamine fluoride.

FIG. 2C is a front view diagram illustrating a tooth during application of laser treatment to a tooth treated with silver diamine fluoride.

FIG. 2D is a front view diagram illustrating a tooth after application of laser treatment to a tooth treated with silver diamine fluoride.

FIG. 2E is a front view diagram illustrating a tooth after application of a glass ionomer cement (GIC) coating.

FIG. 3 is a schematic diagram illustrating a system for removing dental discoloration.

FIG. 4 is a photograph of a temperature sensor embedded in a root canal of a bovine tooth.

FIG. 5A is a photograph of a bovine tooth after application of SDF treatment.

FIG. 5B is a photograph of the bovine tooth of FIG. 5A after application of laser treatment to a portion of the bovine tooth.

FIG. 6A is a photograph of a bovine tooth after application of SDF treatment.

FIG. 6B is a photograph of the bovine tooth of FIG. 6A after application of laser treatment to a portion of the bovine tooth.

FIG. 7A is a CT scan of a bovine tooth after application of SDF treatment.

FIG. 7B is a CT scan of the bovine tooth of FIG. 7A after application of laser treatment to a portion of the bovine tooth.

FIG. 8A is a photograph of a first sample of a bovine tooth before application of SDF treatment.

FIG. 8B is a photograph of the first sample of the bovine tooth of FIG. 8A after application of SDF treatment.

FIG. 8C is a photograph of the first sample of the bovine tooth of FIG. 8B after application of laser treatment to the bovine tooth.

FIG. 8D is a photograph of the first sample of the bovine tooth of FIG. 8C four months after application of the laser treatment.

FIG. 9A is a photograph of a second sample of a bovine tooth before application of SDF treatment.

FIG. 9B is a photograph of the second sample of the bovine tooth of FIG. 9A after application of SDF treatment.

FIG. 9C is a photograph of the second sample of the bovine tooth of FIG. 9B after application of laser treatment to the bovine tooth.

FIG. 9D is a photograph of the second sample of the bovine tooth of FIG. 9C four months after application of the laser treatment.

FIG. 10A is a CT scan of the first sample of the bovine tooth of FIG. 8B before application of the laser treatment.

FIG. 10B is a CT scan of the first sample of the bovine tooth of FIG. 8C after application of the laser treatment.

FIG. 11A is a CT scan of the second sample of the bovine tooth of FIG. 9B before application of the laser treatment.

FIG. 11B is a CT scan of the second sample of the bovine tooth of FIG. 9C after application of the laser treatment.

### DETAILED DESCRIPTION

This disclosure describes devices and method for reducing the silver compounds that cause discoloration following SDF treatment. SDF treatment has significant clinical limitations due to its tendency to cause permanent black discoloration of treated teeth caused by silver compounds, such as silver oxide and silver sulfide. These silver compounds are formed from oxidation of the silver component of SDF after application of SDF. Due to poor cosmetic outcomes, the use of SDF has been limited to treatment of caries in pediatric, geriatric, and medically compromised patient populations that are not able to tolerate more invasive dental procedures.

Conventional enamel whitening agents, such as hydrogen peroxide and carbamide peroxide, can remove many organic discolorations, but are not effective at reducing inorganic silver compounds. Current approaches to treating the discoloration caused by the silver compounds include chemical treatments to remove the discoloration or coverage treatments to cover up the discoloration. For example, potassium iodide solution (KI) applied shortly after SDF treatment may react with silver ions to form a silver iodide precipitate before the silver ions have had an opportunity to arrest caries progression. As another example, glass ionomer cements (GIC) applied over the teeth may cover up and mask the silver compounds, but not enough to prevent discoloration from showing through.

As described herein, discoloration caused by silver compounds after SDF treatment can be removed or reduced while maintaining an anti-microbial action of the SDF treatment. A surface portion of a tooth treated with a silver diamine fluoride (SDF) treatment includes silver compounds, such as silver oxide or silver sulfide, that are generated from a reaction between silver ions and oxygen- and sulfur-containing compounds. To reduce the discoloration caused by these silver compounds, a laser treatment is applied to the tooth to selectively break the chemical bonds of the silver compounds. For example, laser energy applied to the tooth may have a particular wavelength or range of wavelengths that is selectively absorbed by the silver compounds and converts the silver compounds to more neutrally-colored elements or compounds, such as elemental silver and sulfur dioxide. The laser energy may be maintained at a fluence and pulse frequency that maintains a temperature of the tooth below thresholds that may damage the tooth. Following laser treatment, the tooth may be optionally coated with an additional coating, such as a glass ionomer cement (GIC) coating, to additionally mask residual discoloration of the tooth.

By reliably reducing unsightly dark discoloration from SDF treatment, use of SDF may be more acceptable to pediatric and geriatric patients, and may be offered to a larger cohort of adult patients with dental decay. For example, early cavities and root decay that are currently operatively restored could potentially be treated with minimally invasive SDF application instead, followed by laser treatment to remove or reduce discoloration from the SDF application. In this way, SDF treatment used in conjunction with laser treatment may improve outcomes related to tooth decay and sensitivity.

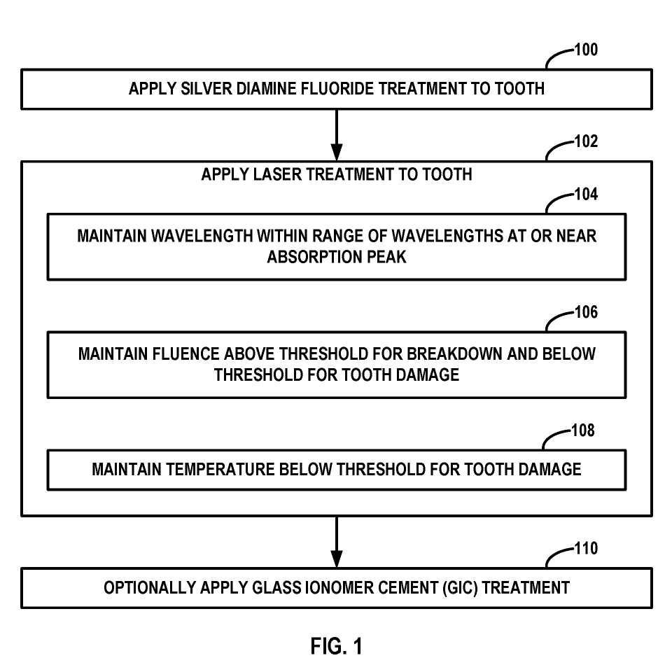

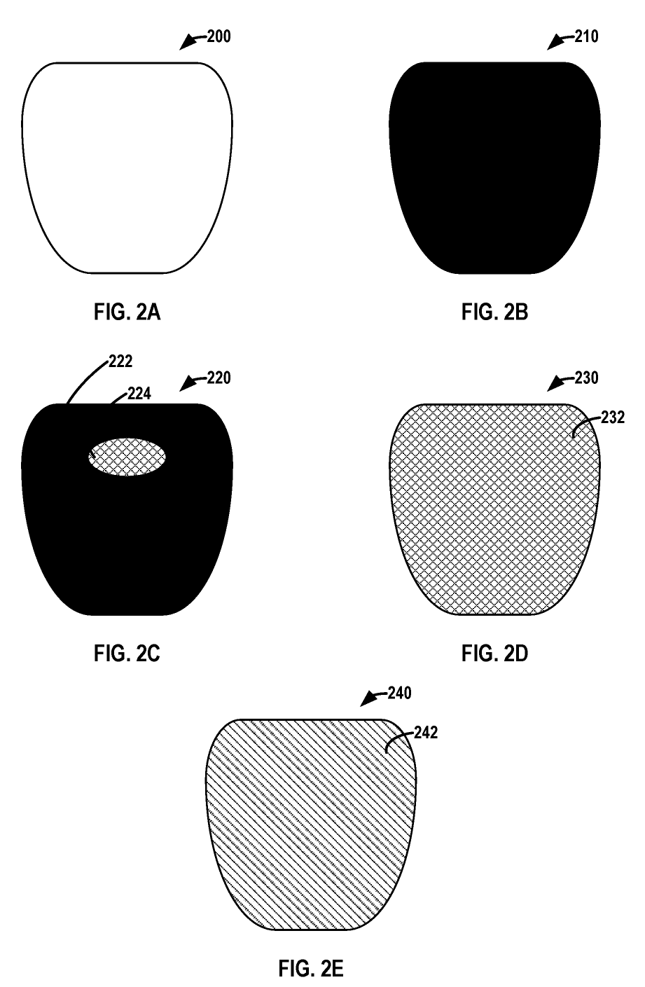

FIG. 1 is a flowchart illustrating an example method for removing dental discoloration, such as dental discoloration caused by SDF treatment. The method of FIG. 1 will be described with respect to FIGS. 2A-2E, which illustrate various phases of treatment of a tooth 200. While FIG. 1 will be described with respect to dental discoloration caused by SDF treatment, the principles described herein may be used to remove other discoloration, such as discoloration caused by other embedded compounds for which a fluence threshold may be lower than a fluence threshold of a tooth. For example, laser treatment may be used to reduce discoloration in teeth caused by other dental therapeutic agents, such as other silver-containing therapeutic agents such as silver nitrate, or other antimicrobial therapeutic agents such as zinc or copper.

FIG. 2A is a front view diagram illustrating tooth 200 prior to treatment with silver diamine fluoride. Tooth 200 may be susceptible to or affected by various dental conditions, such as tooth decay (i.e., dental caries) and tooth sensitivity. While tooth 200 may have some protection from tooth decay and sensitivity, such protection may be limited.

The method of FIG. 1 includes applying the SDF treatment to a tooth (100), such as tooth 200 of FIG. 2A. FIG. 2B is a front view diagram illustrating an SDF-treated tooth 210 after treatment with silver diamine fluoride. Application of an SDF treatment includes applying an SDF solution to one or more surfaces of tooth 200. Silver diamine fluoride (SDF) protects tooth 200 through a combination of chemical reactions and physical processes that allow it to penetrate the tooth structure and exert its therapeutic effects. SDF solution may be applied to the surfaces of tooth 200, such as by using a microbrush or applicator. SDF solution applied to the surfaces coats the outside surfaces and diffuses into the porous structure of the enamel and dentin. Dentinal tubules, which are microscopic channels within the dentin, may facilitate deeper penetration of the solution via capillary action.

As a result of the SDF treatment, SDF-treated tooth 210 includes silver and fluoride ions near the surface of SDF-treated tooth 210 that provide antimicrobial and remineralization actions. For example, silver ions may denature bacterial proteins, disrupt cell walls, and interfere with bacterial DNA replication, leading to bacterial death. Fluoride ions enhance the remineralization of demineralized enamel and dentin and interact with calcium and phosphate in the tooth structure to form fluorapatite (Ca₅(PO₄)₃F), which may be more resistant to acid dissolution compared to hydroxyapatite.

After application of the SDF solution, SDF-treated tooth 210 includes silver compounds as a result of reactions of silver ions with other species in or on the tooth. The silver ions in SDF may react with the tooth structure to form antimicrobial silver compounds. For example, silver ions may react with phosphate ions in the hydroxyapatite to form silver phosphate (Ag₃PO₄), a yellowish compound. However, the silver compounds may include silver oxide and silver sulfide, each of which has a dark color.

The method of FIG. 1 includes applying a laser treatment to the tooth (102). FIG. 2C is a front view diagram illustrating a partially laser-treated tooth 220 during application of laser treatment to a tooth treated with silver diamine fluoride. In the example of FIG. 2C, tooth 220 includes an untreated region 222 and a treated region 224. The laser treatment involves applying laser energy to surfaces of laser-treated tooth 220; this application of the laser treatment selectively breaks the chemical bonds of the silver compounds. Various properties of the laser energy may be selected to enhance removal of the discoloration without damaging surrounding tissue of laser-treated tooth 220.

In applying the laser treatment, the method includes maintaining a wavelength of the laser energy within a range of wavelengths at or near an absorption peak for the silver compounds (104). The silver compounds may have a characteristic absorption spectrum that relates an absorption coefficient (i.e., how much light the silver compounds can absorb) at different wavelengths. Application of the laser treatment includes applying laser energy within a particular wavelength or range of wavelengths with a high absorption coefficient relative to other wavelengths, such as near an absorption peak of the absorption spectrum. The laser energy is selectively absorbed by the silver compounds, such that a relatively large amount of energy is absorbed by the silver compounds compared to other compounds of laser-treated tooth 220. The particular range of wavelengths may be between about 1000 and about 1100 nanometers (nm). In some instances, the particular wavelength is about 1064 nm.

In applying the laser treatment, the method includes maintaining fluence of the laser energy within a threshold range (106). The silver compounds may have a lower fluence threshold above which silver compounds may break down. The fluence threshold for discolored silver compounds may represent a minimum energy per unit area required to break down or ablate the silver compounds, and may be related to an absorption coefficient at the laser wavelength. The lower fluence threshold may be greater than about 0.1 J/cm². However, other compounds of the tooth, such as enamel and dentin, may have a corresponding upper tooth fluence threshold that represents a minimum energy per unit area required to break down or ablate the dentin or enamel. The upper fluence threshold may be less than about 40 J/cm². For particular wavelengths and pulse widths, a threshold fluence of the silver compounds may be below a threshold fluence of tissues of the tooth, such as enamel and dentin. For example, at a 1064 nm wavelength and 5 nanosecond pulse width, the fluence threshold may be about 15 J/cm² for dentin and 20 J/cm² for enamel, while a fluence threshold of silver oxide and silver sulfide may be only 5 J/cm² or less. As such, the fluence of the laser energy may be maintained at or above the fluence threshold that can achieve the breakdown of the silver compounds but below the fluence threshold that would cause damage to the tooth.

In applying the laser treatment, the method includes preventing temperature elevation above physiological temperature from exceeding a threshold value for prolonged periods of time (108). Various tissues of tooth 220, such as the pulp, may be relatively sensitive to increases in temperature, and may begin to experience damage when temperature elevation exceeds a specific threshold value above physiological temperature. The temperature may be maintained below an upper temperature limit corresponding to damage to these sensitive tissues of the tooth. The temperature at the surface of laser-treated tooth 220 that corresponds to a particular temperature at the pulp may be related to various properties of the tooth, such as thickness of the dentin and enamel and the geometry (e.g., the size and the shape) of the tooth. The laser energy may be applied in a manner that limits an increase in temperature in the pulp chamber of laser-treated tooth 220 to below this temperature limit.

In some instances, application of laser treatment includes maintaining a temperature increase at the surface of the tooth below about 5.5 degrees Celsius (℃). To control a temperature of the tooth, application of the laser energy may include pulsing the laser energy. Pulsing the laser energy may limit an amount of heat absorbed by the tooth, thereby reducing a temperature increase. The pulsations may be selected according to a length of time at which the laser energy is applied and a length of time between applications of the laser energy. In some instances, application of the laser energy includes controlling a size of the laser beam. For example, a spot size of the laser may be reduced to limit a temperature increase of the tooth.

FIG. 2D is a front view diagram illustrating a tooth 230 after application of laser treatment to a tooth treated with silver diamine fluoride. In response to application of the laser treatment, the silver oxide and silver sulfide break down to form elemental silver and lighter colored silver compounds. Prior to application of the laser treatment, the tooth has an initial concentration of discolored silver compounds. After the application of the laser treatment, tooth 230 has a final concentration of discolored silver compounds that is less than the initial concentration of discolored silver compounds. In some examples, the laser treatment may be applied to achieve a desired change in color of tooth 230. The laser treatment may be continued until the tooth has a final color index that is cosmetically acceptable, at which point the laser treatment may be stopped.

In the example of FIG. 2D, tooth 230 includes a treated region 232 that includes substantially all of tooth 230. However, in other instances, less than an entire region may be treated. For example, only visible regions of tooth 230 may be treated, or may be more fully treated than other regions.

While steps 100–106 have been described with respect to application of SDF treatment to a tooth, in other instances, methods described herein may be used to removed discolored silver compounds generated by other silver-containing therapeutic agents. For example, silver nitride may be applied to a tooth to generate discolored silver compounds. Alternatively, methods described herein may be used to reduce discolored compounds generated by other therapeutic agents. For example, zinc or copper may form discolored compounds in the teeth capable of being broken down using laser energy having a particular wavelength or range of wavelengths.

In some instances, it may be desired to further enhance a color of the tooth. For example, while laser treatment may substantially reduce SDF-related discoloration of the tooth, the tooth may not be as light colored as desired. As mentioned above, while application of GIC to a tooth treated with SDF may not be capable of covering up the dark discoloration, such application may be capable of covering up any lighter colored discoloration remaining after laser treatment. In such instances, the method of FIG. 1 includes optional application of a glass ionomer cement (GIC) coating or other coating to the tooth (110). FIG. 2E is a front view diagram illustrating a tooth 240 after application of a glass ionomer cement (GIC) coating. Glass ionomer cement is commonly used for its fluoride-releasing properties, biocompatibility, and adhesion to both enamel and dentin. Components of GIC may be mixed to activate the GIC mixture and subsequently applied to surfaces of the tooth using a microbrush or applicator. The GIC mixture may be cured, such as through self-curing or an external curing mechanism (e.g., light). The resulting GIC coating 242 may further protect tooth 240 and lighten a color of tooth 240 beyond the already lightened color that results from the laser treatment.

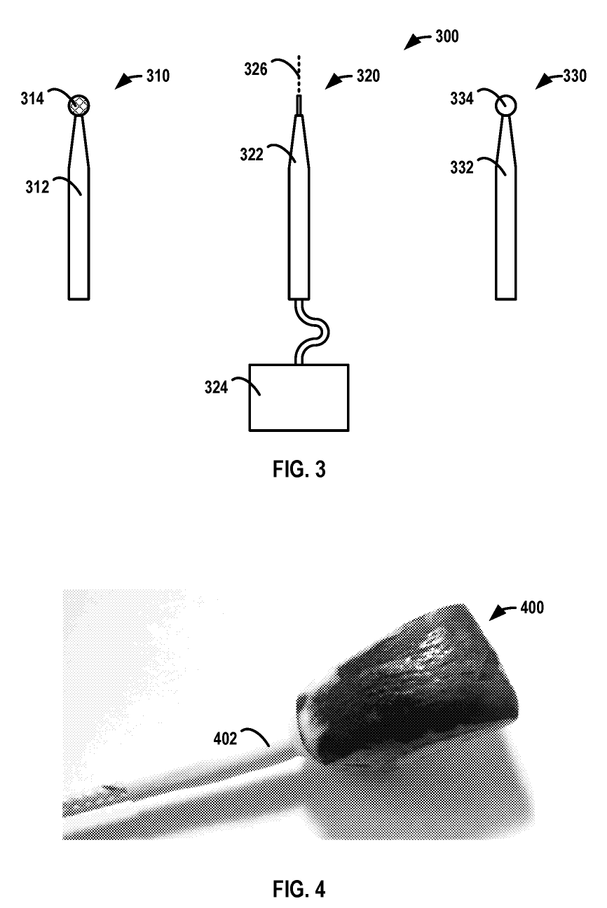

FIG. 3 is a schematic diagram illustrating a system 300 for treating teeth. System 300 includes a silver diamine fluoride (SDF) treatment system 310, a laser treatment system 320, and a glass ionomer cement (GIC) treatment system 330. SDF treatment system 310 is configured to apply an SDF treatment to a tooth. System 310 includes an applicator 312 that applies an SDF solution 314 to one or more surfaces of the tooth.

Laser treatment system 320 is configured to apply a laser treatment to a tooth. Laser treatment system 320 includes a laser treatment device 322 coupled to a power source 324 and configured to apply laser energy 326 to the tooth. A variety of parameters of laser energy 326 may be controlled including, but not limited to, a wavelength, a fluence, a spot size, a pulsation, and the like. To more effectively break the bonds, laser treatment system 320 may be configured to apply laser energy 326 within a particular wavelength or range of wavelengths and a particular fluence. In some examples, the particular range of wavelengths is between about 1000 and about 1100 nanometers (nm), such as about 1064 nm. Laser energy 326 is selectively absorbed by the silver compounds in the tooth. Laser treatment system 320 may be configured to pulse laser energy 326. Such pulsation may limit an increase in temperature of the tooth. Application of laser treatment may include maintaining a temperature increase of the tooth below about 5.5 degrees Celsius (℃).

In some examples, laser treatment system 320 may be specifically configured for applying a laser treatment for reducing discolored silver compounds, such as silver compounds, in teeth that are generated from a silver-containing therapeutic agent. For example, laser treatment device 322 may operate as a single-wavelength laser at relatively low power and feature pulse parameters, such as pulse width, to efficiently target SDF discoloration. Laser treatment device 322 specifically tailored for treating silver-containing therapeutic agent discolored teeth, such as SDF discolored teeth, would have significant advantages over more general use laser treatment devices. By focusing on a single-wavelength corresponding to desired silver compounds, laser treatment system 320 may eliminate the need for expensive and bulky Q-switching equipment that may be required for other clinical applications . Additionally, laser treatment device 322 may operate with pre-programmed settings to enhance ease of use for clinicians. In some instances, laser treatment system 320 may include additional systems configured to aid specifically reduction of discoloration in teeth, such as a cooling system configured to cool the tooth, such as by applying chilled air.

GIC treatment system 330 is configured to apply a glass ionomer cement (GIC) coating to the tooth. System 330 includes an applicator 332 that applies a GIC solution to one or more surfaces of the tooth.

### Experimental Methods

### Experiment 1

Five bovine central incisors were acquired, with samples chosen on the basis of being free from cracks, discoloration, and decay. The selected teeth were sterilized in a 0.1% thymol solution. Samples were partially coated with a clear protective varnish (nitrocellulose-based nail polish) on the mesial, distal, and lingual surfaces, leaving only the buccal surfaces exposed for demineralization.

A 4.5 pH lactic acid solution was prepared, with the acidity adjusted using NaOH. The samples were submerged in the demineralizing solution for two days to simulate enamel demineralization, with the acidic solution changed daily.

A silver diamine fluoride solution (Advantage Arrest, 38% SDF) was applied to four demineralized bovine enamel samples (Sample 1, Sample 2, Sample 3, and Sample 4). The tooth surfaces were first cleaned and dried. For each sample, one drop (approximately 30 μL) of SDF solution was dispensed from the dropper bottle into a dappen dish. A microbrush applicator was used to transfer the SDF solution directly onto the tooth surface, where it was scrubbed for 10 seconds. After allowing the SDF to absorb for 1 minute, the surface was rinsed with deionized water and blotted dry. A fifth sample (Sample 5) served as a control, with no SDF solution applied. All samples were aged for one week in an incubator at 37°C and wrapped in gauze saturated with deionized water. Characterization was conducted using optical microscopy for surface analysis and 3D micro-computed tomography (micro-CT) for mineral density assessment.

FIG. 4 is a photograph of a temperature sensor 402 embedded in a root canal of a bovine tooth 400. Thermocouple temperature sensors were embedded within the root canals of two of the treated bovine teeth to measure temperature changes within the pulp chamber during laser treatment. White thermocouple gel (Arctic Ceramique 2) was extruded into the root canal to establish thermal conductance between the temperature sensor and the pulp chamber.

The SDF discolored teeth were treated with a 1064 nm Nd:YAG laser, capable of operating within a fluence range of 2–12 J/cm². Laser parameters were initially set to 3.9 J/cm² fluence and a 6 mm spot size. Six laser pulses were applied to the upper left quadrant of Sample 1, at which point the black discoloration was reduced to an off-white color. For Sample 2, five pulses were applied over a larger area (upper half of the tooth), until black discoloration was reduced to an orange hue. In Sample 3, the spot size of the laser was narrowed to a 3 mm diameter, keeping the fluence energy the same. For Sample 4, the fluence energy was increased to 6 J/cm² with a narrow 3 mm spot size. Finally, multiple laser pulses (16 pulses, 3.9 J/cm², 6 mm spot size) were applied to Sample 5 for comparison.

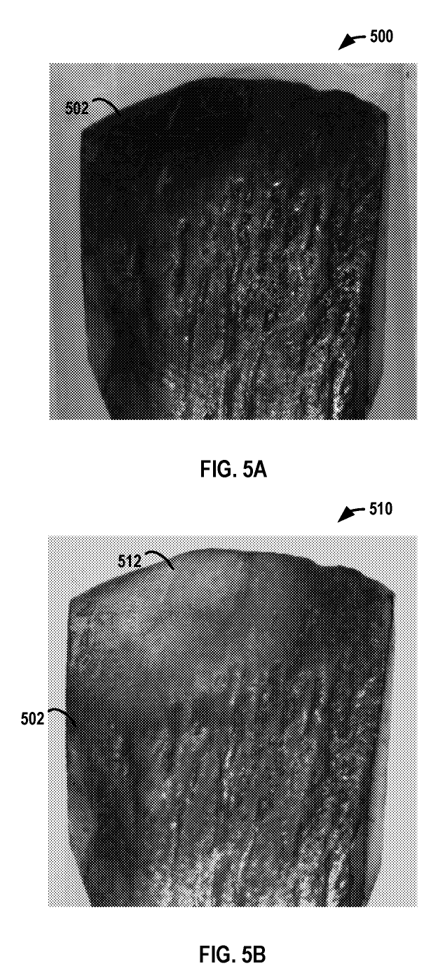

FIG. 5A is a photograph of a bovine tooth 500 of Sample 1 after application of SDF treatment. As seen in FIG. 5A, the bovine tooth 500 treated with SDF exhibited dark, black discoloration 502 before laser treatment. FIG. 5B is a photograph of the bovine tooth 510 of FIG. 5A after application of laser treatment to a portion of the bovine tooth. As seen in FIG. 5B, following the application of 6 laser pulses with 3.9 J/cm² fluence and a 6 mm spot size to a focal region in the top left corner of the tooth, the black discoloration 502 changed to an off-white color 512.

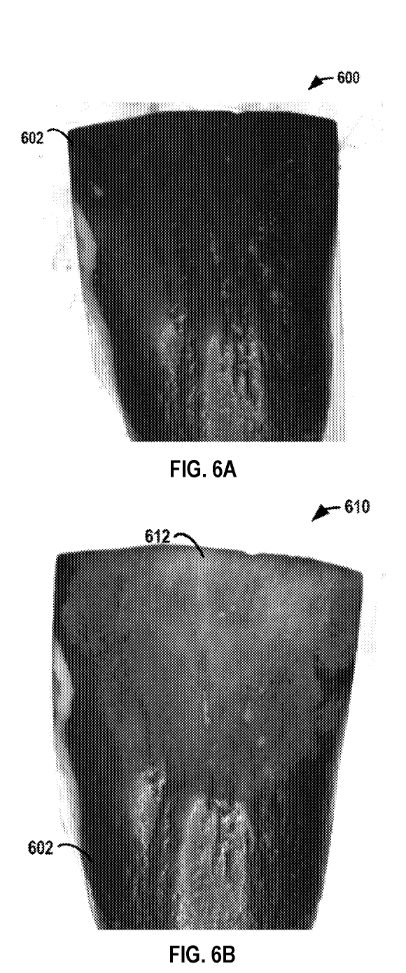

Variations in laser settings were explored across multiple samples, revealing that fluence energy, spot size, and number of laser pulses significantly influenced the final color of the treated region. FIG. 6A is a photograph of a bovine tooth 600 of Sample 2 after application of SDF treatment, while FIG. 6B is a photograph of the bovine tooth 610 of FIG. 6A after application of laser treatment to a portion of the bovine tooth. As seen in FIG. 6B, samples treated with fewer pulses (5) over a larger area, had a more orange appearance 612 from the dark discoloration 602.

When the laser beam spot size diameter was reduced to 3 mm, laser pulses resulted in no observable reduction in silver discoloration, whether at 3.9 J/cm² or 6 J/cm² fluence levels. This suggests that larger spot sizes may be more effective for reducing oxidized silver compounds than narrower ones. In the control sample, no changes in color or surface appearance were observed, even after 16 laser pulses were applied using a 6 mm spot size at 3.9 J/cm².

Further adjustments can be made to one or more aspects of the technique required to determine alternative pulse frequency (e.g., multiple rapid pulses versus single pulses), spot size diameter, and fluence energies to achieve improved cosmetic results for other situations, such as an entire tooth surface.

Assessment of the impact of laser treatment on pulpal temperatures using a thermocouple temperature sensor embedded within the root canal of bovine tooth samples indicated that temperature within the root canal increased by only 1 to 2° C. This is within a safe level, since the dental literature indicates that harm to the pulp and root vasculature occurs only after a temperature increase of at least 5.5° C above physiological levels.

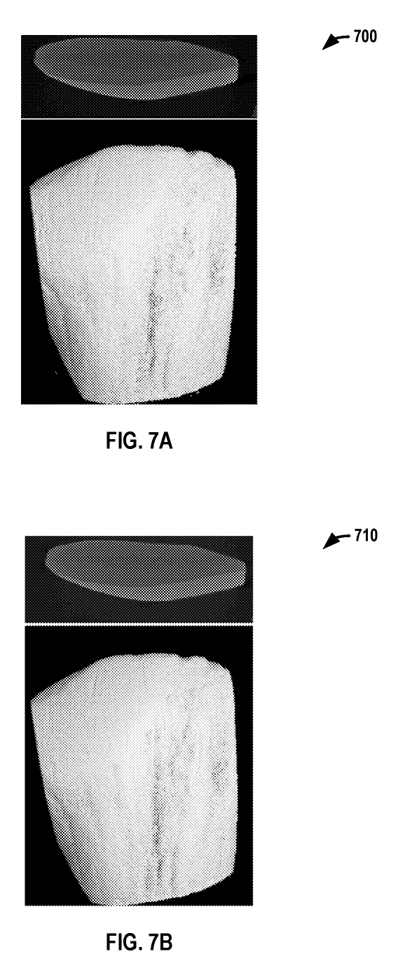

Micro-CT scans, which provide a detailed evaluation of enamel integrity, were performed on samples before and after laser pulsation. The cross-sectional and reconstructed surface topology images showed no observable loss of enamel or decrease in mineral density in the tested samples and the control sample following laser treatment. FIG. 7A is a CT scan of a bovine tooth 700 after application of SDF treatment, while FIG. 7B is a CT scan of the bovine tooth 710 of FIG. 7A after application of laser treatment to a portion of the bovine tooth. As seen in FIGS. 7A and 7B, micro-CT scans before and after laser treatment appear radiographically unchanged.

According to the literature, at λ = 1064 nm, silver tarnish has a lower ablation threshold energy than both dental enamel and elemental silver. As such, it is possible to effectively reduce tarnished and discolored Ag₂O and Ag₂S compounds, without ablating or removing underlying enamel.

After laser treatment, the color of the laser treated regions, where dark silver compounds had been reduced, remained stable. Notably, the laser treated areas did not appear to re-oxidize or revert to a black discoloration, even at three months follow-up, suggesting that the treatment is enduring.

Without being limited to any particular theory, it is postulated that, upon removal of the sulfide and oxide bonds responsible for discoloration, the free silver ions become embedded in or stabilized by the hydroxyapatite crystal network of the enamel. This incorporation could potentially confer long-term antimicrobial resistance and enhanced protection against cavities, without the downside of unsightly discoloration. For example, X-ray diffraction may be used to analyze the structure and properties of the hydroxyapatite-silver network within the enamel following laser treatment.

The 1064 nm Nd:YAG laser effectively reduced discoloration from oxidized silver compounds in SDF-treated teeth. As few as six laser pulses, with 3.9 J/cm² fluence and a 6 mm spot size, were sufficient to convert dark discoloration to a more neutral off-white color. Thermal analysis indicated minimal pulpal temperature increase that is within safe levels, and post-treatment micro-CT scans showed no observable loss of enamel or change in mineral density. Laser treatment holds promise as a solution for reducing SDF-induced discoloration of teeth. Laser parameters may be further improved, and lasers may be utilized to reduce SDF discoloration of other dental tissues, such as dentin, cementum, and root canals.

### Experiment 2

In a preliminary demonstration, the crowns of three extracted bovine central incisors were sterilized in 0.1% thymol solution and then submerged in a pH 4.5 lactic acid demineralization solution for 48 hours, with the acidic solution replaced daily. A microbrush was used to apply one drop (approximately 30 μL) of Advantage Arrest 38% silver diamine fluoride (Elevate Oral Care LLC, West Palm Beach, FL, USA) to the buccal enamel surface of each tooth, where it was scrubbed for 10 seconds. After allowing the SDF to absorb for 1 minute, the bovine teeth were rinsed with deionized water and blotted dry. The lingual enamel surfaces served as untreated controls, with no SDF applied. All bovine samples were wrapped in gauze saturated with deionized water and aged in an incubator at 37 °C for one week, during which time dark discoloration developed.

Prior to laser treatment, baseline characterization was performed. Optical micrographs were obtained at 8× magnification using an Olympus MVX10 stereo microscope (Olympus Corp., Tokyo, Japan), with images taken on a WhiBal G7 gray card (PictureFlow LLC, Phoenix, AZ, USA). Scalebars were inserted using ImageJ software (National Institutes of Health, Bethesda, MD, USA).

Baseline micro-computed tomography (micro-CT) scans were conducted using a Nikon H225 X-Tek XT scanner (Nikon Metrology Ltd., Brighton, MI, USA) to evaluate the initial radiographic appearance of enamel. Micro-CT scans were performed with a tungsten source operated at 115 kV and 75 μA, using 720 projections with 4 frames per projection. Data reconstruction was performed using CTPro 3D (Nikon Metrology, Inc., Brighton, MI, USA), and 2D and 3D image rendering was performed using VGStudio Max 3.4 (Volume Graphics GmbH, Heidelberg, Germany).

Laser treatment was performed on discolored tooth samples with a Quanta Q-Plus C Qswitched Nd:YAG laser (Quanta System S.p.A., Milan, Italy) with 1064 nm and 532 nm settings, and a fluence range of 2–25 J/cm². Sample 1 was treated with 2.6 J/cm² fluence and a 4 mm spot size at a 1064 nm wavelength. Sample 2 was treated with 2.0 J/cm² fluence and 4 mm spot size at a 1064 nm wavelength. Sample 3 was treated with 2.0 J/cm² fluence and 4 mm spot size at a 532 nm wavelength. Laser pulses were also applied to the non-SDF-treated lingual surfaces to serve as controls.

Temperature within the root canal of tooth Sample 1 was monitored during laser application 4 using an Extech MN35 thermocouple and multimeter (Extech Instruments Corp., Nashua, NH, USA), with the thermocouple inserted into the coronal third of the canal. Arctic Ceramique 2 thermocouple gel (Arctic Silver Inc., Visalia, CA, USA) was applied to the probe tip to enable thermal contact with root canal walls.

Optical micrographs and micro-CT scans were obtained following laser treatment using the same settings as at baseline to assess changes in color and radiographic appearance, with additional optical micrographs captured at a four-month follow-up to evaluate longer-term color stability.

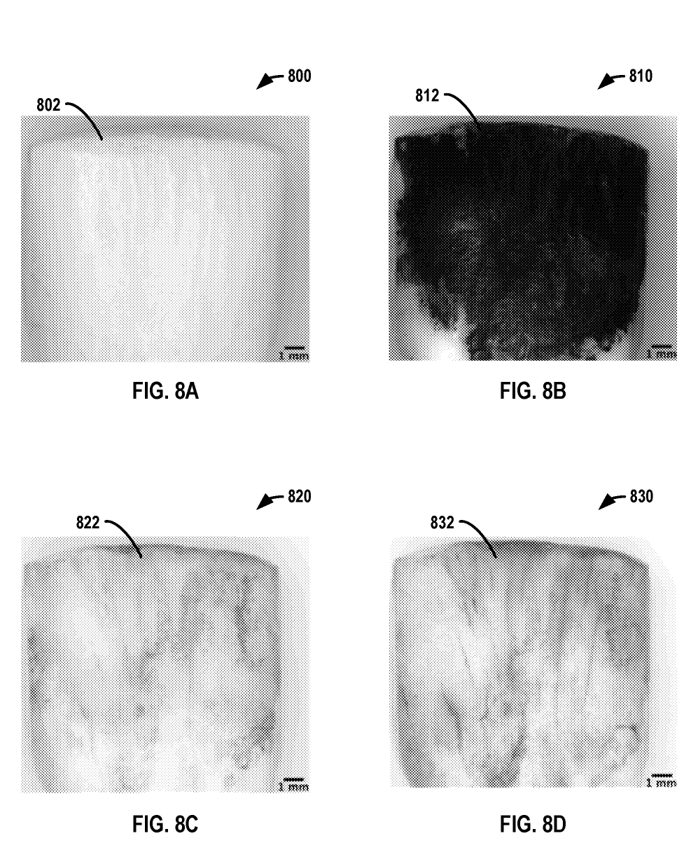

FIG. 8A is a photograph of Sample 1 of bovine tooth 800 before application of SDF treatment. As seen in FIG. 8A, prior to application of the SDF treatment, bovine tooth 800 had demineralized bovine enamel 802 that appeared uniformly white. FIG. 8B is a photograph of the first sample of the bovine tooth 810 of FIG. 8A after application of SDF treatment. As seen in FIG. 8B, the bovine tooth 810 treated with SDF exhibited dark, black discoloration 812 after application of the SDF treatment and one week of aging. FIG. 8C is a photograph of the first sample of the bovine tooth 820 of FIG. 8B after application of laser treatment to the bovine tooth. As seen in FIG. 8C, following the application of 8 laser pulses with 2.6 J/cm² fluence, a 4 mm spot size, and a wavelength of 1064 nm, the black discoloration 812 changed to an off-white color 822. FIG. 8D is a photograph of the first sample of the bovine tooth 830 of FIG. 8C four months after application of the laser treatment. As seen in FIG. 8D, the color of the enamel surface 832 remains stable.

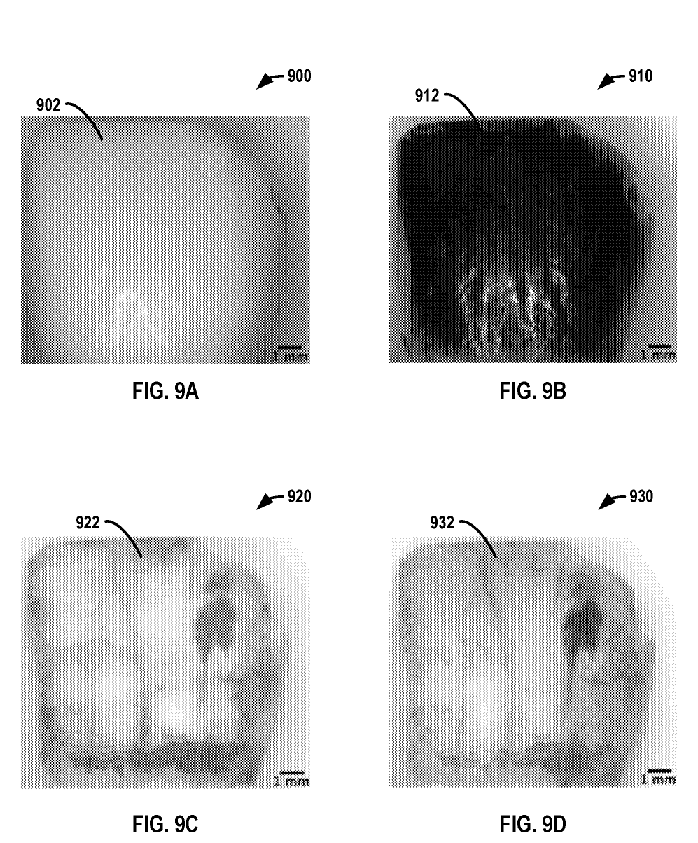

FIG. 9A is a photograph of a second sample of a bovine tooth 900 before application of SDF treatment. As seen in FIG. 9A, prior to application of the SDF treatment, bovine tooth 900 had demineralized bovine enamel 902 that appeared uniformly white. FIG. 9B is a photograph of the second sample of the bovine tooth 910 of FIG. 9A after application of SDF treatment. As seen in FIG. 9B, the bovine tooth 910 treated with SDF exhibited dark, black discoloration 912 after application of the SDF treatment and one week of aging. FIG. 9C is a photograph of the second sample of the bovine tooth 920 of FIG. 9B after application of laser treatment to the bovine tooth. As seen in FIG. 9C, following the application of 13 laser pulses with 2.0 J/cm² fluence, a 4 mm spot size, and a wavelength of 1064 nm, the black discoloration 912 changed to an off-white color 922. Even at this suboptimal low fluence setting, laser treatment of the entire enamel surface eliminated most of the black discoloration, leaving behind gold hues and some small brown discolorations. FIG. 9D is a photograph of the second sample of the bovine tooth 930 of FIG. 9C four months after application of the laser treatment. As seen in FIG. 9D, the color of the enamel surface 932 remains stable.

For Sample 3, the wavelength of the Quanta laser device was switched to 532 nm. Repeated laser pulses were applied with 2.0 J/cm² fluence, but no appreciable improvement in black discoloration was observed.

Control testing, performed by applying laser pulses to non-SDF treated enamel on the lingual surface of bovine teeth, produced no appreciable change in appearance. Pulp chamber temperature was observed to increase by only 2° C during laser treatment, which was below the 5.5°C threshold known to cause pulpal injury.

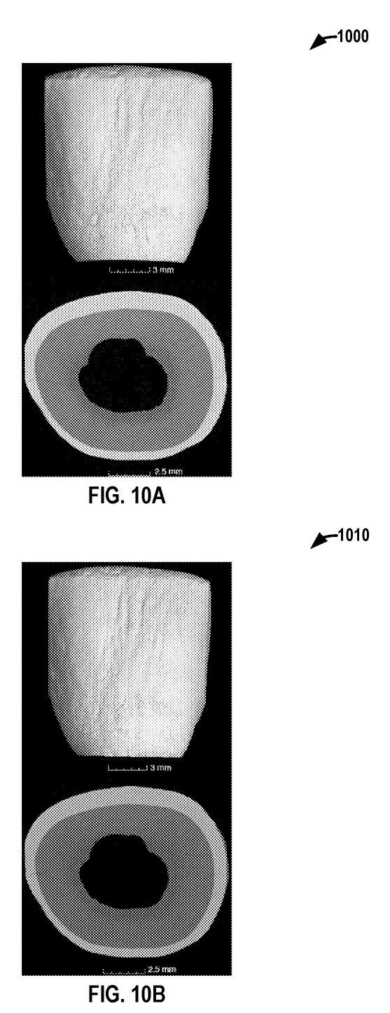

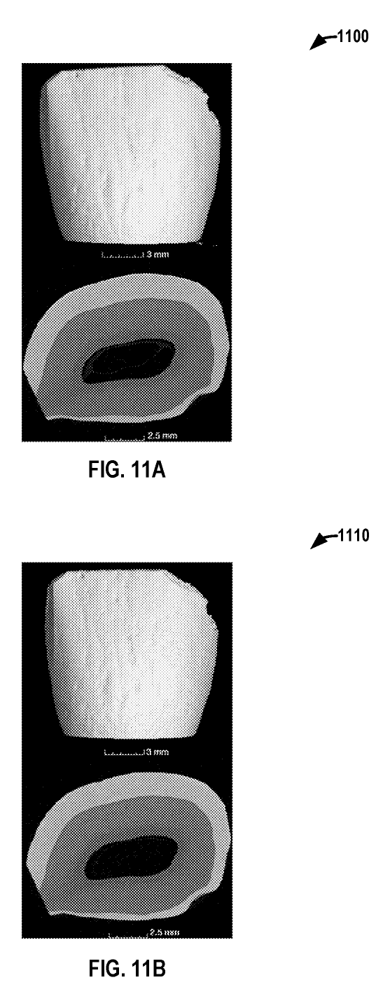

Micro-CT reconstructions, including topographic and horizontal cross-sectional views, showed preservation of enamel following laser treatment. FIG. 10A is a CT scan of the first sample of the bovine tooth 1000 of FIG. 8B before application of the laser treatment, while FIG. 10B is a CT scan of the first sample of the bovine tooth 1010 of FIG. 8C after application of the laser treatment. Similarly, FIG. 11A is a CT scan of the second sample of the bovine tooth 1100 of FIG. 9B before application of the laser treatment, FIG. 11B is a CT scan of the second sample of the bovine tooth 1110 of FIG. 9C after application of the laser treatment. For Samples 1 and 2, which underwent 6 full-surface laser pulsation, the post-treatment micro-CT reconstructions, as illustrated in FIGS. 10B and 11B, respectively, appeared radiographically similar to their respective pre-treatment scans, as illustrated in FIGS. 10A and 11A, respectively.

As illustrated above, application of low-fluence 1064 nm Nd:YAG laser pulses was found to reduce black discoloration on bovine tooth samples, with no appreciable changes in micro-CT appearance and minimal increases in pulpal temperature. Initial observations suggest that appearance of SDF-discolored enamel after laser treatment is fluence-dependent: at 2.6 J/cm², only eight pulses per 4 mm diameter region were needed to achieve an off-white appearance, whereas at a lower fluence setting (2.0 J/cm²), a greater number of pulses (i.e., 13 pulses) were required, with less elimination of discoloration attained. No appreciable effect was observed when laser pulses of 532 nm wavelength were applied, suggesting wavelength specificity for removing SDF discoloration. Notably, the improved appearance of 1064 nm treated enamel remained stable over a four-month observation period, suggesting that removal of black SDF discoloration is enduring.

Without being limited to any particular theories, various mechanisms for the reduction of SDF discoloration may be possible. As one possible mechanism, laser photons may selectively break the chemical bonds of dark-colored silver oxide and silver sulfide compounds, causing elemental silver to be left in the enamel matrix in a less discolored form. As another possible mechanism, the laser energy may cause a localized photothermal effect sufficient to eject superficial tarnish compounds from the tooth surface. The persistence of a gold hue after laser treatment suggests that some silver compounds may remain within the tooth structure.

Example 1: A method for reducing or removing dental discoloration includes applying a laser treatment to a tooth, wherein the tooth includes one or more discolored silver compounds generated from a silver-containing therapeutic agent treatment, and wherein application of the laser treatment selectively breaks chemical bonds of the one or more discolored silver compounds.

Example 2: The method of example 1, wherein applying the laser treatment includes applying laser energy within a particular wavelength or range of wavelengths, and wherein the laser energy is selectively absorbed by the one or more discolored silver compounds.

Example 3: The method of example 2, wherein the one or more silver-containing therapeutic agent treatment comprises a silver diamine fluoride (SDF) treatment.

Example 4: The method of example 3, wherein the particular range of wavelengths is between about 1000 and about 1100 nanometers (nm).

Example 5: The method of any of examples 3 and 4, wherein the particular wavelength is about 1064 nm.

Example 6: The method of any of examples 2 through 5, wherein application of the laser energy includes pulsing the laser energy.

Example 7: The method of any of examples 1 through 6, wherein the one or more discolored silver compounds include silver oxide and silver sulfide, and wherein, in response to application of the laser treatment, the silver oxide and silver sulfide break down to form elemental silver and sulfur dioxide.

Example 8: The method of any of examples 1 through 7, wherein, prior to application of the laser treatment, the tooth has an initial concentration of the one or more discolored silver compounds, and wherein, after the application of the laser treatment, the tooth has a final concentration of discolored silver compounds that is less than the initial concentration of discolored silver compounds.

Example 9: The method of any of examples 1 through 8, wherein application of the laser treatment includes maintaining a fluence of the laser treatment above a fluence threshold of the one or more discolored silver compounds.

Example 10: The method of example 9, wherein the fluence of the laser treatment is greater than about 0.1 J/cm2.

Example 11: The method of any of examples 1 through 10, wherein application of laser treatment includes maintaining a fluence of the laser treatment below a fluence threshold of dentin and enamel.

Example 12: The method of example 11, wherein the fluence of the laser treatment is less than about 40 J/cm2.

Example 13: The method of any of examples 1 through 12, wherein application of laser treatment includes maintaining a temperature increase of the tooth below about 5.5 degrees Celsius (℃).

Example 14: The method of any of examples 1 through 13, further comprising, prior to application of the laser treatment, applying the silver-containing therapeutic agent treatment to the tooth.

Example 15: The method of any of examples 1 through 14, further comprising, after application of the laser treatment, applying a glass ionomer cement (GIC) coating to the tooth.

Example 16: A system for removing dental discoloration includes a laser treatment system configured to apply a laser treatment to a tooth, wherein the tooth includes one or more discolored silver compounds generated from a silver-containing therapeutic agent treatment, and wherein application of the laser treatment selectively breaks chemical bonds of the one or more discolored silver compounds.

Example 17: The system of example 16, wherein the laser treatment system is configured to apply laser energy within a particular wavelength or range of wavelengths, and wherein the laser energy is selectively absorbed by the one or more discolored silver compounds.

Example 18: The system of example 17, wherein the one or more silver-containing therapeutic agent treatment comprises a silver diamine fluoride (SDF) treatment.

Example 19: The system of example 18, wherein the particular range of wavelengths is between about 1000 and about 1100 nanometers (nm).

Example 20: The system of any of examples 18 and 19, wherein the particular wavelength is about 1064 nm.

Example 21: The system of any of examples 17 through 20, wherein the laser treatment system is configured to pulse the laser energy.

Example 22: The system of any of examples 16 through 21, wherein the one or more discolored silver compounds include silver oxide and silver sulfide, and wherein, in response to application of the laser treatment, the silver oxide and silver sulfide break down to form elemental silver and sulfur dioxide.

Example 23: The system of any of examples 16 through 22, wherein, prior to application of the laser treatment, the tooth has an initial concentration of the one or more discolored silver compounds, and wherein, after the application of the laser treatment, the tooth has a final concentration of the one or more discolored silver compounds that is less than the initial concentration of the one or more discolored silver compounds.

Example 24: The system of any of examples 16 through 23, wherein the laser treatment system is configured to maintain a fluence of the laser treatment above a fluence threshold of the one or more discolored silver compounds.

Example 25: The system of any of examples 16 through 24, wherein the laser treatment system is configured to maintain the fluence of the laser treatment at greater than about 0.1 J/cm2.

Example 26: The system of any of examples 16 through 25, wherein the laser treatment system is configured to maintain a fluence of the laser treatment below a fluence threshold of dentin and enamel.

Example 27: The system of any of examples 16 through 26, wherein the laser treatment system is configured to maintain the fluence of the laser treatment at less than about 40 J/cm2.

Example 28: The system of any of examples 16 through 27, wherein application of laser treatment includes maintaining a temperature increase of the tooth below about 5.5 degrees Celsius (℃).

Example 29: The system of any of examples 16 through 28, further comprising a silver diamine fluoride treatment system configured to apply the SDF treatment to the tooth.

Example 30: The system of any of examples 16 through 29, further comprising a glass ionomer cement (GIC) treatment system configured to apply a GIC coating to the tooth.

Various examples of the disclosure have been described. Any combination of the described systems, operations, or functions is contemplated. These and other examples are within the scope of the following claims.

What is claimed is:

1. A method for reducing or removing dental discoloration, the method comprising:
applying a laser treatment to a tooth,
wherein the tooth includes one or more discolored silver compounds generated from a silver-containing therapeutic agent treatment, and
wherein application of the laser treatment selectively breaks chemical bonds of the one or more discolored silver compounds.

2. The method of claim 1,
wherein applying the laser treatment includes applying laser energy within a particular wavelength or range of wavelengths, and
wherein the laser energy is selectively absorbed by the one or more discolored silver compounds.

3. The method of claim 2, wherein the one or more silver-containing therapeutic agent treatment comprises a silver diamine fluoride (SDF) treatment.

4. The method of claim 3, wherein the particular range of wavelengths is between about 1000 and about 1100 nanometers (nm).

5. The method of claim 3, wherein the particular wavelength is about 1064 nm.

6. The method of claim 2, wherein application of the laser energy includes pulsing the laser energy.

7. The method of claim 1,
wherein the one or more discolored silver compounds include silver oxide and silver sulfide, and
wherein, in response to application of the laser treatment, the silver oxide and silver sulfide break down to form elemental silver and sulfur dioxide.

8. The method of claim 1,
wherein, prior to application of the laser treatment, the tooth has an initial concentration of the one or more discolored silver compounds, and
wherein, after the application of the laser treatment, the tooth has a final concentration of discolored silver compounds that is less than the initial concentration of discolored silver compounds.

9. The method of claim 1, wherein application of the laser treatment includes maintaining a fluence of the laser treatment above a lower fluence threshold of the one or more discolored silver compounds.

10. The method of claim 1, wherein the fluence of the laser treatment is greater than about 0.1 J/cm².

11. The method of claim 1, wherein application of the laser treatment includes maintaining a fluence of the laser treatment below an upper fluence threshold of dentin and enamel.

12. The method of claim 1, wherein the fluence of the laser treatment is less than about 40 J/cm².

13. The method of claim 1, wherein application of laser treatment includes maintaining a temperature increase of the tooth below about 5.5 degrees Celsius (℃).

14. The method of claim 1, further comprising:
prior to application of the laser treatment, applying the silver-containing therapeutic agent treatment to the tooth; and
after application of the laser treatment, applying a glass ionomer cement (GIC) coating to the tooth.

15. A system for removing dental discoloration, the system comprising:
a laser treatment system configured to apply a laser treatment to a tooth,
wherein the tooth includes one or more discolored silver compounds generated from a silver-containing therapeutic agent treatment, and
wherein application of the laser treatment selectively breaks chemical bonds of the one or more discolored silver compounds.

16. The system of claim 15,
wherein the laser treatment system is configured to apply laser energy within a particular wavelength or range of wavelengths,
wherein the laser energy is selectively absorbed by the one or more discolored silver compounds,
wherein the silver-containing therapeutic agent treatment comprises a silver diamine fluoride (SDF) treatment, and
wherein the particular range of wavelengths is between about 1000 and about 1100 nanometers (nm).

17. The system of claim 16, wherein the particular wavelength is about 1064 nm.

18. The system of claim 16, wherein the laser treatment system is configured to pulse the laser energy.

19. The system of claim 15, wherein the laser treatment system is configured to maintain a fluence of the laser treatment above a lesser of:
a lower fluence threshold of the one or more discolored silver compounds; or
about .1 J/cm².

20. The system of claim 15, wherein the laser treatment system is configured to maintain a fluence of the laser treatment below a lesser of:
an upper fluence threshold of dentin and enamel; or
about 40 J/cm².
